(19) United States
(12) Patent Application Publication
(10) Pub. No.: US 2016/0339213 A1
(43) Pub. Date: Nov. 24, 2016

(54) METHOD AND SYSTEM FOR SECLUDING A BODY VESSEL

(71) Applicant: David Martin, Atlanta, GA (US)

(72) Inventor: David Martin, Atlanta, GA (US)

(21) Appl. No.: 14/718,865

(22) Filed: May 21, 2015

(51) Int. Cl. A61M 25/10; A61K 31/095; A61K 33/14; A61K 31/08

(52) U.S. Cl. CPC A61M 25/1002; A61M 25/1011; A61K 31/08; A61K 31/095; A61K 33/14; A61M 2025/1052; A61M 2025/105

(57) ABSTRACT

A method for secluding a body vessel at two points is disclosed wherein a chemical agent is maintained at a first point in the vessel for a period of time and wherein a chemical agent is delivered to a second point in the vessel for a period of time. The method can employ a double or triple balloon catheter and can be used for various treatments, including for sclerotherapy of veins.

### BACKGROUND

For certain medical conditions, it may be necessary or desirable to seclude (i.e., to close off, collapse, or significantly narrow) a body vessel such as a vein or artery. One situation in which seclusion may be desirable is in the treatment of varicose veins, which are swollen, twisted, or enlarged veins that may be visible under a patient's skin. By closing off the varicose vein, blood ceases to flow in the varicose vein and is naturally redirected to healthy veins. Over time, the closed-off vein may be completely absorbed into surrounding tissue.

There are several techniques currently in use for secluding a blood vessel such as a varicose vein. Examples of these techniques include surgery, heat ablation, and chemical treatment.

Surgically, veins may be subjected to a seclusion procedure known as ligation. During ligation, a small incision may be made near the target vein and the vein may be tied off. The ligated vein may be left in place and absorbed into surrounding tissue, as noted above. Alternatively, the ligated vein may be removed by a process known as “stripping” the vein. The surgical treatment of veins in this manner is sometimes referred to as phlebectomy.

The surgical treatment of varicose veins is generally effective, but may carry certain risks and disadvantages. The procedure is relatively invasive compared to other varicose vein treatment methods, and accordingly may be painful for some patients. Surgical treatment of varicose veins also carries a risk of nerve injury, may require the use of general anesthesia and an overnight hospital stay, and may require a relatively long recovery time.

Accordingly, other types of vein treatment have been developed. These treatments generally involve damaging the walls of the vein, which causes the vein walls to collapse, close, or narrow.

For example, in heat ablation treatment, a heat source (typically a laser or radio frequency transmitter) may be inserted into the vein through a catheter. Upon reaching a target area of the vein, the heat source may be turned on for a predetermined period of time, which damages the target area of the vein and causes scar tissue to form on the inner walls of the vein. The build-up of scar tissue closes the vein.

Problematically, the same heat that damages the vein can also damage surrounding tissue and nearby nerves. It can also cause skin burns and blood clots, and may not be appropriate for all types of veins.

The vein walls can also be damaged chemically in a procedure known as sclerotherapy. In sclerotherapy, a chemical known as a sclerosing agent may be injected into the vein. The sclerosing agent may damage the walls of the vein and cause the vein to narrow or close.

However, in order to be effective the sclerosing agent needs to remain in contact with the inside walls of the target area of the vein for some time (e.g., up to one minute). This is difficult to achieve using conventional schlerotherapy procedures, because the sclerosing agent may be quickly washed away by the flow of blood through the vein. As a result, the sclerosing agent may be diluted and flow to other portions of the body, and hence the sclerosing agent may not be sufficiently effective to close the vein upon an initial application. Accordingly, patients may need several treatment sessions with one or more injections of sclerosing agent applied in each session.

In order to address these issues, a new sclerotherapy treatment method called catheter-directed foam sclerotherapy (“CDFS”) has recently been employed. In this method, a catheter is inserted into the vein and moved to the target site. The sclerosing agent is injected into the vein through the catheter in the form of a foam. Because the agent is a foam, it is relatively more difficult for the blood flow to dilute and remove the sclerosing agent. Therefore, as compared to conventional sclerotherapy, CDFS allows the sclerosing agent to be present at the target site for a relatively longer period of time, in a relatively larger concentration. Nonetheless, the sclerosing agent will still be washed away from the target site due to the flow of the blood in the vein, so repeated treatments may remain necessary.

### BRIEF OVERVIEW OF THE DRAWINGS

FIG. 1 depicts an exemplary apparatus suitable for use with embodiments of the present invention, in a pre-deployed form.

FIG. 2 depicts the apparatus of FIG. 1 in a deployed form.

FIG. 3 depicts the apparatus of FIG. 1 with an optional inflated inner balloon.

FIG. 4 depicts a body vessel with an identified area to be closed or secluded.

FIG. 5 is a flowchart describing a method of secluding a body vessel at two points, according to an exemplary embodiment.

### DETAILED DESCRIPTION

Exemplary embodiments provide a method and system for secluding a body vessel. According to one embodiment, the vessel may be analyzed to determine a length along which the vessel should be secluded. One end of the length may be identified as a first (starting) point, and a second end of the length may be identified as a second (ending) point. Using a suitable apparatus, a chemical agent may be delivered to treatment areas along the identified length, starting at the first point. The chemical agent may be maintained in the treatment areas for a predetermined period of time sufficient to allow the chemical agent to either chemically damage the vessel walls, or to induce a biological reaction causing the walls to narrow or close (or both). The process may be repeated along the identified length until the second point is reached.

In the case of a vein (such as a varicose vein), the above-described method may allow the vein to be closed or collapsed, thereby limiting the flow of blood through the vein. After some time has elapsed, the vein may be absorbed into surrounding tissue.

The above-described method may be employed in connection with a specially-designed catheter for delivery of the chemical agent to a precise location for a precise period of time. In comparison to conventional techniques, the described methods are less invasive, less damaging of nearby nerves, tissue, and skin, have a reduced recovery time, and are less likely to need multiple applications.

An exemplary apparatus suitable for use with the exemplary invention will now be described. The particular apparatus described below is intended to be exemplary, and one or ordinary skill in the art will recognize that the present invention may be practiced using a different apparatus. Suitable apparatuses may include more or fewer parts than those described below.

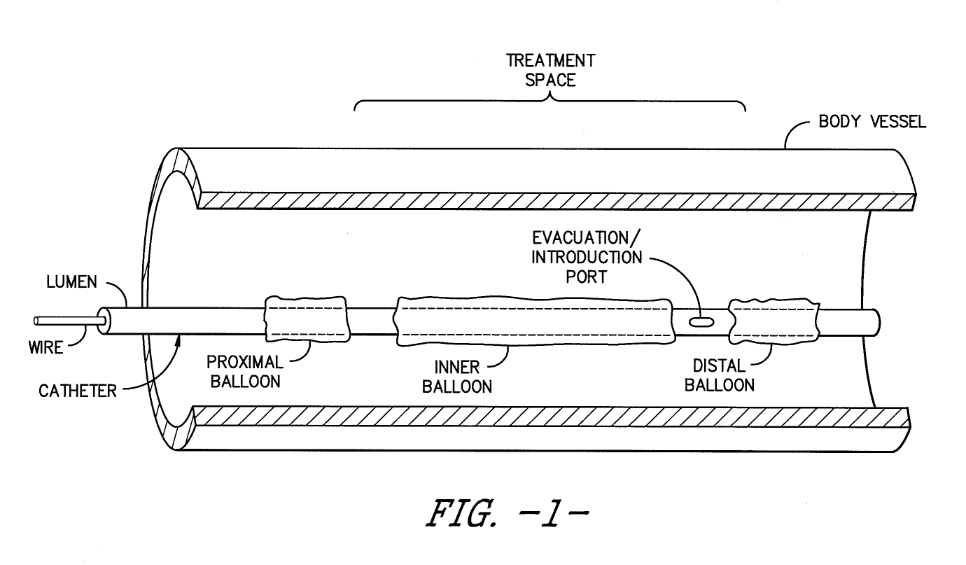

FIG. 1 depicts an exemplary apparatus in the form of a catheter. The catheter may include a lumen, a distal balloon and a proximal balloon defining a treatment space, and an optional inner balloon between the distal balloon and the proximal balloon.

The lumen may be a hollow, flexible tube suitable for guiding the catheter into position inside the body vessel (e.g., via a guidance wire). The lumen may also be used for delivering and/or removing one or more fluids to the treatment space. The lumen's diameter and length may vary depending on the application and the body vessel into which the catheter is inserted. For example, in a case where the body vessel to be secluded is a vein, the lumen's diameter may be typically up to 1 millimeter, or sufficient to carry the guidance wire. The uninflated balloons have typical diameters of between about 3 and about 5 millimeters, but the present invention is not limited to a particularly-sized lumen or balloon.

The distal balloon and proximal balloon may be affixed to the lumen at certain points to define a treatment space between the balloons. When inflated, the interior sides of the distal balloon and proximal balloon (i.e., the sides facing internally towards each other) define the outer limits of the treatment space. The distal and proximal balloons may be sized so that, when inflated, the outer ends of the balloons contact the interior surface of the body vessel and form a seal, preventing fluids from entering or leaving the treatment space. For example, the distal and proximal balloons may typically be 5-10 mm in diameter when inflated to accommodate the volume of the filling air or fluid, and typically 1-2 cm in length. However, again, the present invention is not limited to balloons of any particular size. The distal and proximal balloons may have any suitable shape when inflated, such as spherical, cylindrical, or oblate.

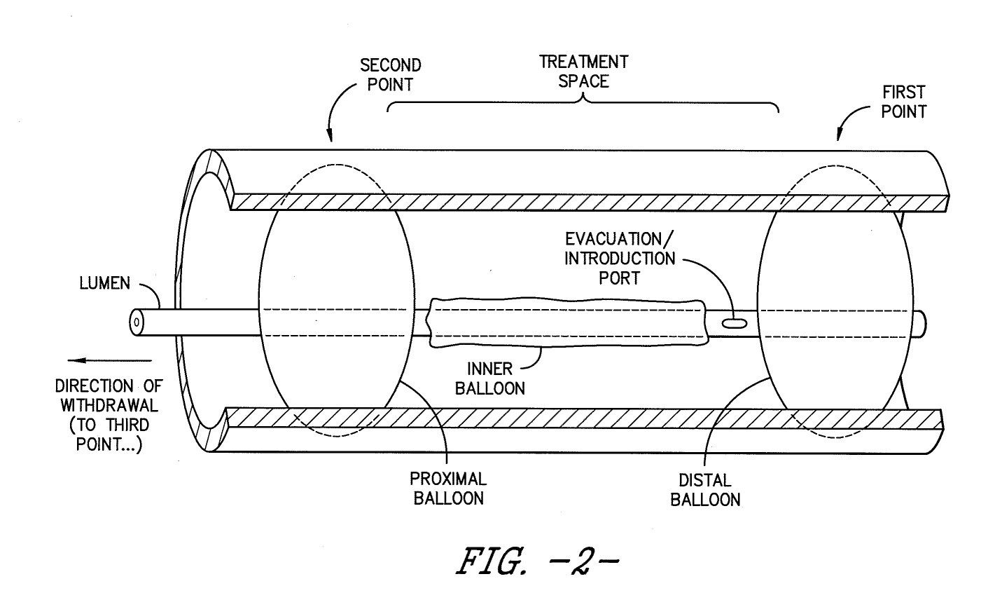

An example of the catheter after being deployed (with inflated proximal and distal balloons) is depicted in FIG. 2.

The area between the inflated proximal balloon and the inflated distal balloon may define a treatment space. The treatment space represents the area into which a chemical agent may be introduced in order to seclude the body vessel between a first point and a second point. The treatment space may be sized according to the application; according to an exemplary embodiment, the treatment space may be 7-15 cm long, but the treatment space necessary will depend upon the particular application and anatomy of the patient.

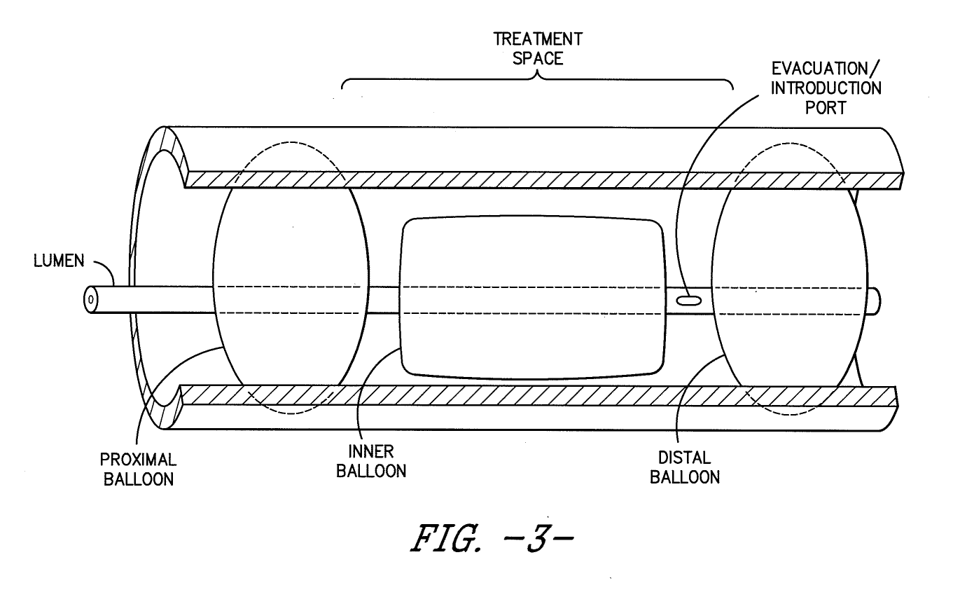

Optionally, the catheter may be provided with an inner balloon. The inner balloon may be sized so that it fills some of the volume inside the treatment space, but does not abut against the walls of the vessel. When the inner balloon is inflated (as shown in FIG. 3), the inner balloon occupies some of the area inside the treatment space while still allowing fluids to reach the walls of the body vessel in the treatment space. Thus, the inner balloon effectively reduces the available volume inside the treatment space, which allows the treatment space to be filled using a smaller amount of chemical agent.

The inner balloon, the distal balloon, and the proximal balloon may be inflated through the lumen of the catheter. The balloons may be inflated using air, or with another suitable fluid.

The length of the inner balloon may be dependent upon the length of the treatment space. In some embodiments, a small amount of space may be left between the inflated distal/proximal balloons and the inflated inner balloon. According to one embodiment, this space may be 1-10 mm in length, but the present invention is not limited to any particular sizes.

Also in the area of the catheter that occupies the treatment space, the lumen may be provided with one or more ports for evacuating fluids and introducing fluids. In the example provided in FIGS. 1-3, a single port is provided for introducing and evacuating fluids, although more ports may be provided in other embodiments. The port may be used, for example, to evacuate blood or other fluids from the treatment space in order to provide an empty area for the chemical agent to occupy, and to prevent the chemical agent from being diluted. The chemical agent may be introduced through the port and may optionally be evacuated from the port after the treatment space has been sufficiently treated with the chemical agent.

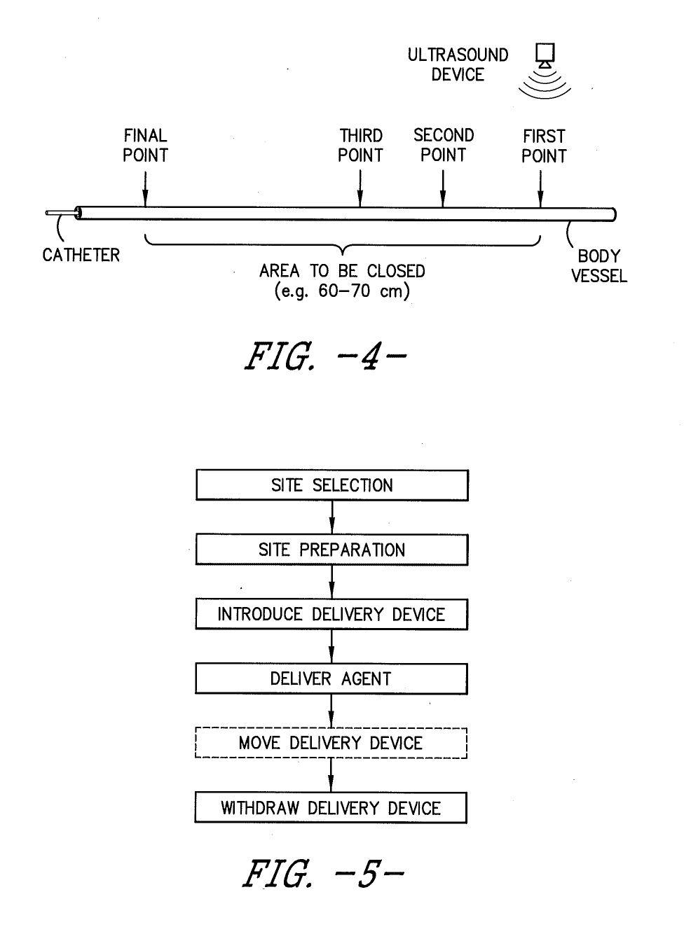

An exemplary method for secluding a body vessel is now described with reference to FIGS. 4 and 5, using the above exemplary apparatus as an example.

First, a target site for two-point seclusion may be identified. The target site may be a body vessel, which in some embodiments may be a vein (such as a varicose vein). Advantageously, the present invention is suitable for use with some types of body vessels which are not well-suited to alternative treatments, such as vulvar varicose veins, perforator veins, and veins of the superficial system.

Using suitable diagnostic techniques, a user may identify a length of the body vessel between a starting point (the “first point” in FIG. 4) and an ending point (the “final point” in FIG. 4) which should be subjected to seclusion. For a typical varicose vein, the length to be secluded may be, for example, 60-70 cm. If the length between the starting point and the ending point is too long to be treated in the treatment space of the catheter, then the length may be subdivided by additional points, and the treatment area of the catheter may be repositioned during the procedure.

Once identified, the target site may be prepared for seclusion. In order to reach the target site with the catheter, an incision in the patient's skin may be made. The body vessel may then be opened at the location of the incision, and a guide wire may be inserted into the opening in the body vessel.

The guide wire may be threaded through the body vessel under guidance of a visualization device. In one embodiment, the visualization device may be an ultrasound device, as depicted in FIG. 4. Ultrasound may be particularly well-suited to positioning the apparatus in exemplary embodiments, because it is not invasive and does not require special equipment to be deployed on the catheter or guide wire.

Once the guide wire has reached the first point for treatment (as indicated by the ultrasound device), the lumen of the catheter may be placed over the guide wire, and the catheter may be threaded into the body vessel. The catheter may be pushed along the length of the guide wire until the distal balloon is in a suitable location at the first point of the body vessel, as indicated by the ultrasound device.

After the catheter is in position, the proximal and distal balloons may be inflated in order to secure the catheter in place and isolate the treatment area. Any fluids remaining in the treatment area (e.g., blood or other body fluids) may optionally be evacuated through the evacuation port. If desired, the inner balloon may be inflated to reduce the available fluid volume inside the treatment area.

A chemical agent may then be delivered through the introduction port. The chemical agent may be an agent known to chemically damage the body vessel into which the catheter has been introduced, thereby causing the body vessel to narrow or close. For example, the chemical agent may be a sclerosing agent typically used in sclerotherapy, such as polidocanol, sotra-decol, hypertonic saline, or any other agent that is capable of damaging the vessel in the context of the sclerosing effect.

Alternatively or in addition, the chemical agent may be an agent known to elicit a biological response from the body vessel into which the catheter has been introduced. Such an agent may be selected so as to induce the biological reaction substantially immediately after the catheter is withdrawn from the treatment site (i.e., the body vessel closes or narrows around the catheter as the catheter is withdrawn from the body vessel). In this way, the vein can be caused to immediately close following application of the chemical agent (as compared to traditional sclerotherapies, in which the vessel may take several days, or even several weeks, o close following application of the sclerosant).

The chemical agent may be delivered to the introduction port of the lumen and from there into the treatment space. Because the proximal and distal balloons create an isolated area inside the treatment space, the chemical agent can be maintained in contact with the vessel walls in the treatment space for a predetermined period of time. In one embodiment, the predetermined period of time may be substantially one minute, or longer. Such an embodiment compares favorably to traditional sclerotherapy, where the sclerosant may be diluted or washed away by the flow of fluid in the vessel shortly after introduction of the sclerosant. After the predetermined period of time, the chemical agent may optionally be evacuated through the evacuation port.

If the treatment space fully encompassed the identified area to be closed, the catheter may now be withdrawn. The body vessel is now secluded between two points (e.g., the first point and the second point in FIG. 4).

If the area to be closed extends beyond the current treatment length, on the other hand, the catheter may be partially withdrawn in order to reposition the treatment space at a new location. In the example of FIG. 4, the catheter may initially be positioned so that the treatment space lies between the first point and the second point. Following application of the chemical agent between these points, the catheter may be repositioned so that the distal balloon is now positioned at the second point, and the proximal balloon is now positioned at the third point. The catheter may be repositioned under guidance of the ultrasound device.

This process may be repeated until the treatment space encompasses the final point (e.g., the proximal balloon is placed at the final point). In one embodiment, the length of area to be treated may be 60-70 cm.

After the chemical agent has been applied along the length of the area to be closed, the catheter may be withdrawn from the body vessel. The inner balloon may be deflated, and any remaining agent may optionally be evacuated through the evacuation port. The outer balloons may be deflated, and the catheter may be withdrawn through the original incision. The original incision in the body vessel and/or the skin may be closed (e.g., with sutures).

Following treatment, a patient will typically be capable of walking immediately and can return home right away (i.e., the patient does not need to remain in a hospital overnight). The body vessel that was treated will generally be secluded immediately (as opposed to conventional sclerotherapy, which may require some time following treatment and/or multiple treatments in order to seclude the vessel), and the vessel may be absorbed into surrounding tissue over a period of several months. The patient will typically be scheduled for a follow-up visit to verify that the vessel has been properly secluded and absorbed. If a problem is noted at the follow-up, the patient may undergo another round of treatment using the above-described exemplary embodiments, or may be treated using a different method.

In contrast to conventional methods, including surgery, heat ablation, and conventional sclerotherapy (including catheter-directed foam sclerotherapy), the above-described techniques may be less invasive and may require less recovery time. A general anesthetic typically does not need to be deployed in exemplary embodiments, relying instead on a local anesthetic. This reduces the risks of the procedure and further decreases required recovery time. Moreover, the above-described embodiments are associated with a reduced risk of nerve and skin damage, and reduced pain in recovery.

It is noted that, as used herein, the term “body vessel” includes any lumen or other similar region in a body, such as blood vessel or intestine. Although specific examples are provided herein with reference to veins, one of ordinary skill in the art will recognize that the present invention is not limited to these particular examples, but rather may be employed in any suitable body vessel.

The term “seclusion” refers to the narrowing, collapsing, or closing off of a body vessel. Accordingly, seclusion is distinct from therapies intended to open or widen a vessel, and from therapies intended to prevent the vessel from narrowing. The term “two-point seclusion” refers to secluding the body vessel at two points with a narrowed, collapsed, or closed space between the points.

As used herein, the article “a” is intended to include one or more items. Where only one item is intended, the term “a single” or similar language is used. Further, the phrase “based on,” as used herein is intended to mean “based, at least in part, on” unless explicitly stated otherwise. In addition, the term “user”, as used herein, is intended to be broadly interpreted to include, for example, an electronic device (e.g., a workstation) or a user of an electronic device, unless otherwise stated.

No element, act, or instruction used in the description of the invention should be construed critical or essential to the invention unless explicitly described as such. It is intended that the invention not be limited to the particular embodiments disclosed above, but that the invention will include any and all particular embodiments and equivalents falling within the scope of the following appended claims.

1. A method for secluding a body vessel at two points, comprising:
identifying a first point and a second point at which the body vessel should be secluded;
delivering a chemical agent to the first point inside the body vessel;
maintaining the chemical agent at the first point for a first predetermined period of time to seclude the body vessel at the first point;
delivering the chemical agent to the second point inside the body vessel; and
maintaining the chemical agent at the second point for a second predetermined period of time to seclude the body vessel at the second point.

2. The method of claim 1, wherein the body vessel is a vein.

3. The method of claim 2, wherein the vein is a varicose vein

4. The method of claim 2, wherein the vein is a vulvar varicose vein

5. The method of claim 2, wherein the vein is a perforator vein or a vein of the superficial system.

6. The method of claim 2, wherein secluding the first point and secluding the second point causes the vein to be closed to a flow of blood.

7. The method of claim 1, wherein the first point and the second point are secluded by chemically damaging the body vessel.

8. The method of claim 1, wherein the first point and the second point are secluded using sclerotherapy by application of a sclerosing agent.

9. The method of claim 8, further comprising maintaining the sclerosing agent at the first point for a period of at least one minute.

10. The method of claim 8, wherein the sclerosing agent is introduced by a catheter, and the sclerosing agent is selected so as to induce a biological reaction that secludes the body vessel substantially immediately after withdrawing the catheter from the first point or the second point.

11. The method of claim 1, wherein secluding the first point comprises positioning a catheter at the first point and secluding the second point comprises moving the catheter through the body vessel from the first point to the second point.

12. The method of claim 1, wherein the first point and the second point are secluded using a catheter, and the catheter is positioned at the first point and the second point using ultrasound.
